(19) United States
(12) Patent Application Publication
(10) Pub. No.: US 2013/0190712 A1
(43) Pub. Date: Jul. 25, 2013

(54) WASHABLE BABY DIAPER WITH CENTRAL INTERIOR OPENING

(71) Applicant: Ada Vaughan, (US)

(72) Inventor: Ada Vaughan, (US)

(21) Appl. No.: 13/543,869

(22) Filed: Jul. 8, 2012

Related U.S. Application Data
(60) Provisional application No. 61/589,061, filed on Jan. 20, 2012.

(51) Int. Cl. A61F 13/505; A61F 13/493

(52) U.S. Cl. 604/385.14; 604/385.15

(57) ABSTRACT

An exemplary embodiment of a washable and reusable baby diaper with central interior opening. The preferred embodiment of said diaper is comprised of two main components including an approximately hourglass shaped two layer outer containing element, and a contoured absorbent soaker pad with a plurality of fabric layers sewn together. The outer fabric of the containing element is laminated on its interior surface to provide dryness protection. A unique longitudinal central interior opening provides a means for keeping said soaker pad in place during wear, containing solid waste, and allowing for ease of washing and use.

This application claims the benefit of provisional patent application No. 61/589,061 filed 2012 Jan. 20 by the present inventor.

### FEDERALLY SPONSORED RESEARCH

Not Applicable

### SEQUENCE LISTING OR PROGRAM

Not Applicable

### BACKGROUND

1. Field of Invention

This invention relates to washable and reusable baby diapers.

2. Prior Art

Mothers have been using washable diapers for centuries. In 1955 R. May patented an adjustable cotton diaper and waterproof covering using U.S. Pat. No. 2,703,577. This is an early example of the 2-component washable diaper consisting of an absorbent inner layer (usually cotton), and waterproof or water-resistant outer layer (plastic, rubber, or vinyl). Diaper services were widely used to launder the cotton material, and households took weekly deliveries of clean cotton diapers. With more women working outside the home, and US consumers demanding a more convenient option, in the 1960s disposables became the dominant diapering modality. By 1980, washables had nearly disappeared, except in the poorest of conditions. However, in the last twenty years, several inventors have sought to improve on the washable diaper. In order to save money, reduce household waste, and avoid chemical exposure to their baby, many parents are turning back to washable diapering options. Several patents have been issued in this area. Inventions such as “Reusable Diapers” Published Application No. US 2008/0065039 by Labit et al. presented 2-component diapering with an envelope, or pocket holding the absorbent material, and a containing element with stretchy sides and rows of snaps down the front to adjust the size of the diaper.

While more recent patents provide significant advantage over the squares of cotton and plastic pants we saw in the 1960s, they still pose challenges in implementation. There are several disadvantages to the adjustable size pocket or envelope style diaper covers heretofore presented:
a) This structure requires the absorbent material to be removed from the pocket prior to laundering the diaper. Since modern washable diaper users (mothers) typically wash their own diapers at home, this becomes a dirty and time-consuming chore. In addition to pulling a soiled and foul-smelling fabric pad out of each diaper prior to washing, they must also take the time to re-assemble the laundered pad and cover prior to next use.
b) Adjusting the rise of the diaper with rows of snaps is not only unattractive, but does a poor job of fitting babies at the small and large end of the stated size range. Extra bulk from snapping down the rise makes for an awkward roll of fabric at the front of the diaper for smaller babies. Nothing is done to adjust the size of the leg nor waist openings, so leaks can occur on smaller babies.
c) Since pocket or envelope style diapers have an attached layer of material against the baby's skin, the entire diapering system must be changed each time, even if only slightly wet.
d) Pocket style diapers have only a single leg elastic to contain solid human waste once the diaper is soiled.
e) Snap closures are difficult to use, and add bulk to the diaper. They may also be uncomfortable pressing into the skin when the baby is on his/her stomach.
f) Hook and loop closures become less and less effective over time with repeated laundering. Fuzz, lint, and hair get trapped in the hooks, and the closure tabs curl up, eventually rendering the closure (and diaper) unusable.
g) Soaker pads with many layers of material are apt to trap bacteria, soap residue, and odors. These thick pads also take significant time and energy to tumble dry in home machines, frequently an hour or more.

### SUMMARY

In accordance with one embodiment a washable and reusable baby diaper comprises an approximately hourglass shaped water-resistant containing element with removable closure tabs and inner fabric layer with elongated central interior opening. Used inside the containing element is a contoured, multi-layer absorbent soaker pad.

### DRAWINGS—FIGURES

In the drawings, closely related figures have the same number but different alphabetic suffixes.

FIG. 1 shows a side and interior view of the complete diaper with the front end of the soaker pad outside of the central interior opening.

FIG. 2 shows the front view of the diaper closed, as it would be when worn by a baby.

FIGS. 3a and 3b show a flat view of the interior of the diaper. FIG. 3a shows the soaker pad outside of the central interior opening. FIG. 3b shows the soaker pad inside the central interior opening, as it would be while in use.

FIGS. 4a to 4d show the construction of the multi-layer soaker pad.

### REFERENCE NUMERALS

100 Modern Washable Diaper with Unique Interior Opening
105 Oblong longitudinal central interior opening
115 Exterior waterproof or water-resistant fabric
110 Wing portion wraps around the baby's sides to form front closure
120 Elasticized back portion of waist
125 Elasticized leg opening
130 Elasticized channels of central interior opening
135 Loop material across front of diaper
140 Multi-layer soaker absorbent pad
145 Loop material on inside of diaper Wing
150 Hook-hook double sided removable closure tab
155 Attachment point securing Soaker (140) optional, depending upon embodiment
160 Fabric strip attaches to Soaker (140) and diaper at (155), depending upon embodiment
165 Top layer material to wick moisture down to interior absorbent layers
170 Absorbent material layers
175 Top section of Soaker pad which includes (165) and two of (170)
180 Bottom section of Soaker pad which includes two layers of (170)
185 Stitching through all layers of Soaker pad at front end
190 Stitching through all layers of Soaker pad and fabric strip (160) when present at rear end

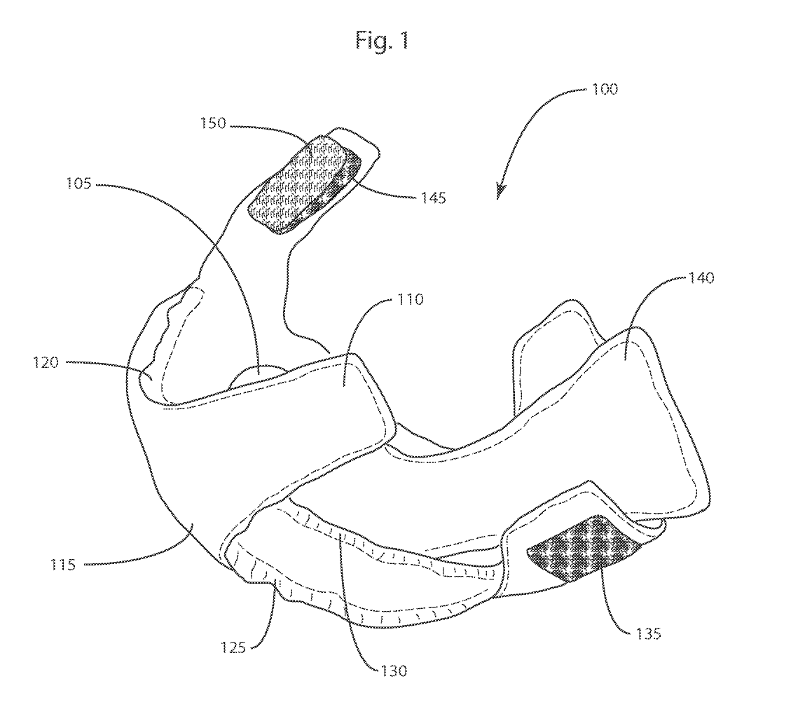

### DETAILED DESCRIPTION—FIGS. 1 to 3b—PREFERRED EMBODIMENT

One embodiment of the diaper is illustrated in FIG. 1 (side/interior view). The diaper 100 is hourglass shaped and has a containing element constructed of exterior, water-resistant fabric 115 and also has absorbent layers in the form of a soaker element 140 to hold liquid waste. This figure shows the soaker pad 140 partially outside of the longitudinal central interior opening 105. When worn, the container element wraps around the baby's body to contain solid and liquid waste, closing over the abdomen with wings 110. The side openings hug the baby's leg with an elasticized edge 125. In addition, the longitudinal central interior opening 105 has an elasticized edge to further contain solid waste inside the diaper. The back edge is elasticized 120 for a closer fit to the body and to contain waste. The diaper closes with hook-and-loop materials 135, 145, 150 to stay on the baby's body. A strip of loop material across the front of the diaper 135 as well as loop patches on the wings 145 allows the wings 110 to be secured to the front with hook/hook closure tabs 150. Two closure tabs 150 are used to hold said wings 110 closed, and allow for placement anywhere along the front loop strip 135 for adjustable fit. The closure tabs 150 have hook material on both sides, and can be completely removed from the diaper, which is recommended when machine washing. The closure tabs 150 are typically shaped as a rectangle with rounded corners, but could also be round, oval, or any decorative shape with sufficient surface area to grab the loop elements 135, 145.

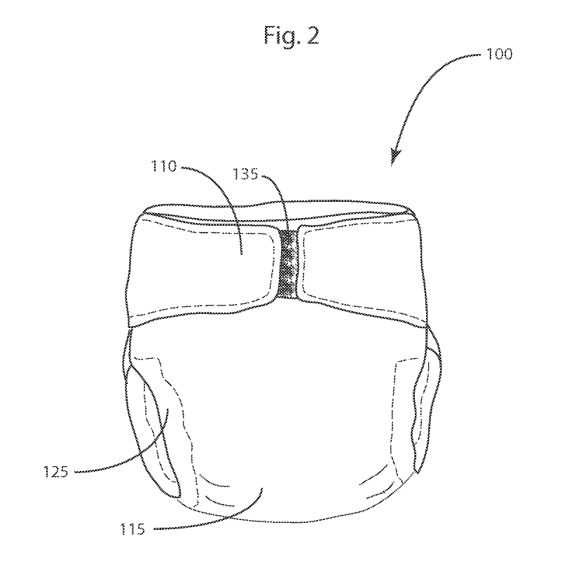

FIG. 2 shows the diaper 100 as it would be worn by a baby in its closed position. The diaper wings 110 are positioned for comfort and security at any place along the loop strip 135. The exterior fabric 115 is designed to keep solid and liquid waste from soaking through the diaper's layers to the baby's clothing. The exterior fabric is soft on the outside and laminated with a waterproof but breathable coating on the inside. Elasticized openings 125 hug the baby's legs to prevent leaks.

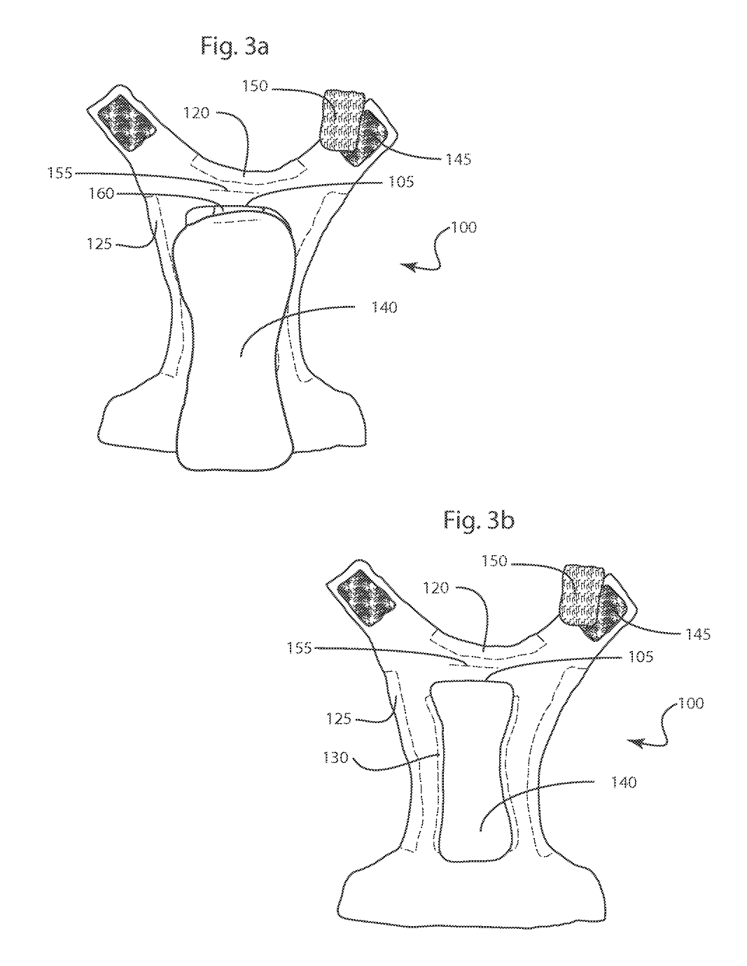

FIGS. 3a and 3b illustrate a flattened view of the diaper interior. Both figures show a closure tab 150 partly removed from the patch of loop material 145 on the diaper wing. FIG. 3a shows the absorbent soaker pad 140 outside of the central interior opening 105. FIG. 3b shows the absorbent soaker pad 140 tucked inside the central interior opening 105 as it would be when in use on a baby. In this embodiment, the soaker pad 140 is connected to the inside of the diaper shell by a small strip of fabric 160 stitched in at attachment point 155. Attachment of the soaker pad 140 to the diaper interior is optional, and could be omitted, or accomplished with a removable fastening such as hook and loop or snaps. The central interior opening 105 is designed to keep the soaker pad 140 from shifting around during wear, while still allowing it to easily come out of the container element during the machine washing process.

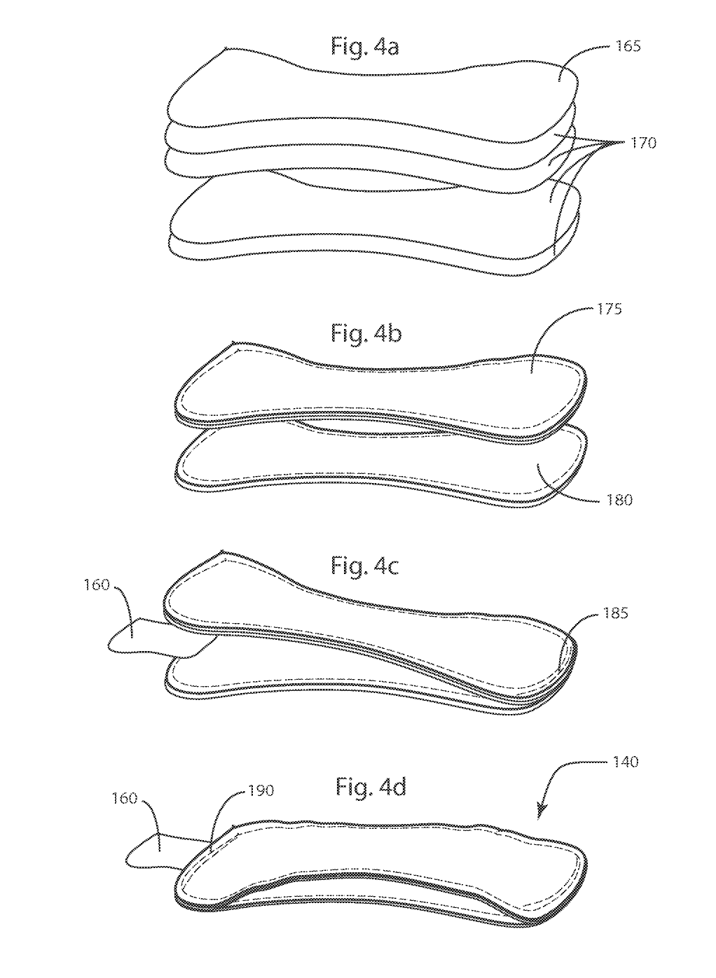

FIGS. 4a to 4d illustrate the construction of the multi-layer absorbent soaker pad 140. FIG. 4a shows the five separate layers of fabric used in the preferred embodiment. The top layer of fabric 165, which is designed to be worn closest to the baby's skin, and is of a material with hydrophobic, wicking action (i.e. suedecloth or fleece) as a means of drawing urine away from the skin. The underneath layers 170 are of an absorbent material (i.e. microterry, microfiber, cotton, hemp, or bamboo blends). This embodiment shows four layers of absorbent material, however two or more layers could be used for absorbency. In FIG. 4a the top set of layers consists of one wicking layer 165 and two absorbent layers 170. The bottom set of layers consists of two absorbent layers 170. FIG. 4b shows the top set of layers 175 and bottom set of layers 180 being stitched together around their perimeter. Any secure method of stitching could be used, although serging or tight overlock is preferred for a smooth and ravel-free edge. FIG. 4c shows the front end of the soaker pad being secured, stitching through both sets 175, 180 of fabric layers 185. Optional fabric strip 160 may be secured at the rear end of the soaker pad. In this embodiment it is secured in FIG. 4d with stitching 190. FIG. 4d shows the final appearance of the multi-layer soaker. This embodiment allows water, detergent, and air to better circulate during machine washing and drying due to the opening between the top and bottom sets of layers.

### Advantages

a) the design and construction of this embodiment is very similar to using a disposable diaper, thus making consumers comfortable with its ease and similarity of use.

b) Attaching the soaker pad to the interior of the diaper saves time in assembly after washing, thus making it easier for moms to use.

c) Removable hook closures significantly extend the life of the hook material, and therefore the usable life of the diaper.

d) The elongated interior opening keeps the absorbent layers from moving around inside the diaper, and contains solid waste much more securely than in prior art.

e) The elongated interior opening allows moms to wash the diaper without pulling out the soaker pad, since it will exit on its own during machine washing.

f) The multi-layer absorbent soaker pad pulls moisture away from baby's skin thereby reducing irritation and resulting rash.

g) The multi-layer absorbent soaker pad is constructed such that it will get cleaner (i.e. trap less soap residue and bacteria between layers) and dry faster than other absorbent pads.

h) The water resistant material is coated on the interior to maintain a soft exterior feel while remaining highly functional.

i) The exterior fabric can be produced in a multitude of attractive colors and printed designs for a fashionable look favored by moms.

j) Since the entire diaper is washable, use of this product will reduce household trash and resulting landfill waste.

### Conclusion, Ramification, and Scope

Accordingly, the reader will see that the washable diaper presented in this application is designed to offer parents an alternative to the cost, waste, and potential irritants associated with disposable diapers. This embodiment gives parents an easy-to-use washable diaper that performs in a superior way to prior patents by extending the life and functionality, decreasing maintenance and assembly time, and simplifying the cloth diapering process. The key advantages to parents are
Ability to keep potentially irritating chemical fragrances, absorbent beads, and non-breathable plastics out of their baby's nursery
Decreasing cost of diapering. Disposable diapers may cost a family $2,500 or more for one child from birth to potty training A set of washable diapers may be used again and again, for $400 or less.
Reducing the household trash and associated landfill waste generated by disposable diapering products. Many families would like to have an alternative to this problem.
   Additional advantages of the design presented in this application include
Easier to use and maintain than prior art
Ability to produce with decorative prints and colors on the exterior
Soft fabric feel (not crunchy or plasticky feeling)
Multi-layer soaker provides high absorbency
Soaker stays in place during use, yet easily self-ejects during washing
Double leg gussets formed by elastic channels provide protection against leaks

Although the description above contains many specificities, these should not be construed as limiting the scope of the embodiment but as merely providing illustrations of some of the presently preferred embodiments. For example, the diaper wings may be secured with plastic snaps or other materials; the size may be varied to accommodate from newborn to toddler-sized babies; the absorbent pad has a plurality of layers depending upon absorbency level desired; different fabrics may be used; the soaker pad may or may not be attached to the diaper interior; etc.

Thus the scope of the embodiment should be determined by the appended claims and their legal equivalents, rather than by the examples given.

1. A washable baby diaper, comprising:
(a) An approximately hourglass shaped containing element composed of a plurality of fabric layers including exterior water resistant fabric and interior moisture wicking fabric, and
(b) an interior layer with longitudinal central interior opening, and
(c) an associated absorbent pad with a plurality of fabric layers, and
(d) a plurality of adjustable and repositionable closures, and
(e) elasticized leg openings, back edge, and central interior opening as a means of providing better fit to the wearer, and
(f) whereby solid and liquid waste will be well contained and easily cleaned out of said diaper.

2. The diaper of claim 1 wherein repositionable hook closures 1d are removable from said diaper as a means of keeping said closures from being machine washed and therefore wearing out.

3. The diaper of claim 1 wherein absorbent pad 1c is attached to the interior of said diaper as a means of saving reassembly time after machine washing and drying said diaper.

4. The diaper of claim 1 wherein absorbent pad 1c is separable from the interior of said diaper as a means of reusing the outer portion of said diaper without laundering.

5. The diaper of claim 1 wherein absorbent pad 1c is of a contoured shape as a means of fitting closely and comfortably to the baby's body.
